(19) United States
(12) Patent Application Publication
(10) Pub. No.: US 2014/0073891 A1
(43) Pub. Date: Mar. 13, 2014

(54) INSTRUMENT FOR MEASURING CARBON MONOXIDE POISONING OF HUMANS USING IN VIVO NIRS TECHNOLOGY

(71) Applicants: Hugh Franklin STODDART, Groton, MA (US); Hugh Adam STODDART, Harvard, MA (US)

(72) Inventors: Hugh Franklin STODDART, Groton, MA (US); Hugh Adam STODDART, Harvard, MA (US)

(73) Assignee: NEUROPHYSICS CORPORATION, Shirley, MA (US)

(21) Appl. No.: 14/080,079

(22) Filed: Nov. 14, 2013

Related U.S. Application Data
(63) Continuation-in-part of application No. 11/739,880, filed on Apr. 25, 2007, now abandoned.

(51) Int. Cl. A61B 5/1455

(52) U.S. Cl. CPC A61B 5/14551
USPC 600/328; 600/322

(57) ABSTRACT

Spectral absorption based non-invasive procedure for determination of blood constituents utilizing in vivo NIRS (Near-Infrared Spectrum) technology, which is the measurement of the near-infrared absorption spectrum within a region of the living human body for the purpose of identifying tissue and blood components and their concentrations and more particularly to novel applications and methodology for determining the optical response, measurements and calculations relating to the concentrations of individual chromophores in the bloodstream and particularly to the level of CO chromophores in the tissues of an animal or human being.

### RELATED APPLICATIONS

This application is a continuation-in-part and claims the benefit of U.S. application Ser. No. 11/739,880 filed Apr. 25, 2007 entitled An Instrument for Measuring Carbon Monoxide Poisoning of Humans Using In Vivo NIRS Technology which is incorporated herein by reference in its entirety.

### FIELD OF THE INVENTION

The present invention relates to in vivo NIRS (Near-Infrared Spectroscopy) technology, which is the measurement of the near-infrared absorption spectrum within a region of the living human body for the purpose of identifying tissue and blood components and their concentrations and more particularly to novel applications and methodology for determining the optical response, measurements and calculations relating to the concentrations of individual chromophores in the tissues and particularly to the level of CO chromophores in the tissues of an animal or human being.

### BACKGROUND OF THE INVENTION

Transillumination is at the most basic level, the transmission of light through tissues of the body. A common example is the transmission of light from a flashlight through fingers, producing a red glow easily observed in a darkened room. This is due to the fact that red blood cells absorbed other colors of the beam and favor transmission of the red component. Except for blood, human tissue is surprisingly translucent to near infrared light. The effect can be easily seen using a red laser “pointer” as used for slide presentations to probe one's own tissues in a dark room. The light is transmitted through thin parts (cheek, ear, fingers, etc.) and a halo of “back-scattered” light can be observed from all tissues.

Transillumination is a major application of visible light in medicine. It is mainly used by pediatricians to shine light into the bodies of infants and observe the amount of scattered light. Since their skeleton is not fully calcified, light can easily penetrate tissues. Common examples of diagnosis are: Light penetrates to the inside of the skull of the infant. If there is an excess of cerebrospinal fluid (CSF), light is scattered to different parts of the skull, producing patterns characteristic to hydrocephalus. The device used in this operation is a Chun gun that uses a 150 watt projection bulb as a light source. Another example is where bright light penetrates the thin front chest wall and reflects off the back chest wall to indicate the degree of pneumothorax. To treat it, a physician will insert a needle attached to a syringe into the area of collapse to remove the air between lungs and chest wall, causing the lung to reinflate.

The features and forms of transillumination have also been used for the examination and analysis of tissue. Specifically the in vivo examination of human female breast tissue, and also to the general field of optical light propogation and response technology in the examination of human female breast anatomy by use of optical response observation, response measurements and characterization, and including spectral response by way of transmissibility, reflection and scattering aspects and relationships.

Blood consists of 55% plasma (water, dissolved salts and proteins), 45% red cells and a smattering of white cells. Virtually all the oxygen carried by the blood is bound to hemoglobin in the red cells (erythrocytes). There are about 280 million red-colored hemoglobin molecules within each of the 10¹³ erythrocytes circulating in the blood. Each molecule of hemoglobin can carry as many as four molecules of oxygen and hemoglobin can transport over 70 times more oxygen than can be simply dissolved in the blood. Bright red hemoglobin that is 100% saturated with oxygen is called oxyhemoglobin (HbO). Hemoglobin without oxygen is called deoxyhemoglobin (or simply Hb) and is much darker and bluish.

At sea level pressure (760 mm Hg) the 21% oxygen in air has a partial pressure (pO₂) of 160 mm of mercury. If air is bubbled through blood, the hemoglobin becomes about 100% saturated with oxygen. The saturation of hemoglobin is not linear with the partial pressure of oxygen but “s” shaped, rising very slowing at low pO₂ and then more rapidly as the oxygen tension approaches 20 mm where the slope becomes steepest. By 60 mm the saturation is already up to 90 “SAT” (percentage of full saturation). This relationship, called the “oxygen dissociation curve” is not fixed, but highly affected by other constituents such as carbon dioxide, acidity (pH) and temperature. At 30 mm oxygen tension, a 10 mm increase in CO₂ tension will lower oxygen saturation by about 4 SAT units as will a 0.1 pH decrease or 2 degree C. temperature increase.

Hemoglobin takes on a maximal load of oxygen over a wide range of atmospheric oxygen partial pressures and carries it to the tissues of the body where oxygen is being consumed and the O₂ tension is low. As tissue pO₂ falls, the hemoglobin is forced to give up some of its oxygen making it available to the tissue. The greater the local metabolism, the lower the tissue oxygen tension. In rapidly metabolizing tissue, the O₂ tension falls to the steep part of the dissociation curve and much more oxygen is released by the hemoglobin.

The oxygen-depleted venous blood flows back to the lungs where the hemoglobin again becomes saturated with oxygen from the alveoli of the lungs.

In order to transport oxygen to cells, there needs to be at least a 20 mm (Hg) gradient between the oxygen-using part of the cell, i.e. mitochondria and the hemoglobin in the capillaries.¹ When pO₂ falls to this value little oxygen can be transferred even though the hemoglobin is still 25% saturated.² ¹Landis E M, Pappenheimer J R, “Exchange of substances through the capillary walls”, Chapter 29, Handbook of Physiology, Circulation, Vol. II (Hamilton WF, editor), 1963, American Physiological Society, Washington, D.C.²Anaerobic degradation of glucose to lactate (glycolysis) is an emergency way of producing chemical energy (ATP) when the oxygen supply is cut off. In fact, red cells are always anaerobic. They do not use oxygen but depend entirely on glycolysis for their chemical energy.

Much of the foregoing can be explained by the existence of two species of hemoglobin called “relaxed” hemoglobin (Hb^{R}) and “tense” or “tight” hemoglobin (Hb^{T}).³ Although these molecules have the same chemical composition, they are dimensionally slightly different. The difference that we are mostly interested in is the distance of the iron atom in the flat “heme” groups from the effective hemoglobin surface. In Hb^{T}, all four iron atoms are 6×10⁻⁵ microns deeper than Hb^{R}. The result is that oxygen can reach an iron atom in the Hb^{R} form allowing it to bind tightly while in Hb^{T} the affinity for oxygen binding is about 100 times less, only about 1% of its affinity for Hb^{R}. ³Perutz M F, “Hemoglobin Structure and Respiratory Transport”, 1978, Scientific American 239 #6 (Dec) pp. 92-125. (Note that Perutz and Kendrew shared the Nobel prize in chemistry in 1962 for their discovery of the structures of hemoglobin and myoglobin).

Hemoglobin is either in one form or the other—there is nothing in between. The hemoglobin molecule literally “snaps” between the two forms. Deoxyhemoglobin consists of Hb^{T} molecules and oxyhemoglobin consists of Hb^{R}. What happens in between explains a lot.

Assume deoxyHb^{T} is at zero pO₂ and gradually increase the oxygen tension. At low pressures, very little oxygen will be absorbed because of the very low oxygen affinity of tight hemoglobin. This is the “toe” of the dissociation curve. As pO₂ increases, some of the Hb^{T} molecules will begin to add a molecule of oxygen. As pressure continues to increase, more hemoglobin molecules will bind an oxygen molecule. At some point, the pressure will be high enough for some hemoglobin molecules to bind a second oxygen. As the number of bound oxygen molecules increase so does the tendency for hemoglobin to snap from Hb^{T} to Hb^{R}. Once the change takes place to the relaxed form of the hemoglobin molecule, its affinity for oxygen is so great that it quickly fills all four of its heme sites. One of the factors driving the conversion may be the tension of the bound oxygen molecule pulling on the iron atom in the heme group. Some hemoglobin molecules convert on adding the second oxygen and others wait until the third. Hb^{T} cannot exist with four oxygen molecules. Other coexisting conditions affect the conversion point. As pO₂ increases, a crescendo of clicking Hb^{T} to Hb^{R} conversions occurs giving rise to the steep part of the oxygen dissociation curve.

The reverse process occurs. Arterial hemoglobin is almost entirely oxyhemoglobin (Hb^{R}). As it enters the capillary beds of the tissues, it encounters falling pO₂ which starts the conversion of some Hb^{R} to Hb^{T}. When a hemoglobin molecule snaps to the tight form, its four heme groups retract and the remaining oxygen gets ejected making it available for tissue metabolism. The transition from Hb^{R} to Hb^{T} is strongly enhanced by H⁺ ions entering the red cells. A single H⁺ ion can trigger the conversion of a molecule of hemoglobin to the tight form and eject its remaining oxygen. This is the mechanism for the “Bohr” effect.

Besides H⁺ ions, increasing concentrations of Cl ions, CO₂ and DPG⁴ all favor Hb^{T}. Most of the agents that drive hemoglobin to the tight form also raise the equilibrium constant for the first oxygen bound, which exaggerates the “s” shape of the dissociation curve and increases its maximum slop (Hill's coefficient”). ⁴The release of oxygen is enhanced by 2,3-Diphosphoglycerate (DPG). There is about one molecule of DPG for every molecule of hemoglobin in red cells. More DPG both decreases the hemoglobin affinity for oxygen and steepens the slope of the “s-shaped” dissociation curve. Taken together, the effect is to force hemoglobin to release even more oxygen to tissues as pO₂ falls. It turns out that DPG plays a direct role in tightening the hemoglobin molecule and forcing its conversion to Hb^{T}.

Agents driving the hemoglobin the other way towards Hb^{R} are, besides oxygen, carbon monoxide (CO) and nitric oxide (NO). Hemoglobin has even more affinity for these gases than for oxygen. A heavy smoker will lower the O₂ concentration in the arterial blood by 20%. Increasing temperature favors Hb^{R} and decreases the maximum slope of the dissociation curve.

Except for small pressure gradient effects between capillary blood pO₂ and mitrocondra pO₂, and variations in the dissociation curve as discussed above, the hemoglobin oxygen saturation of the venous blood draining the capillaries is indicative of tissue oxygen partial pressure.⁵ To the extent that large vessels act as “sinks” for photons, NIRS technology favors capillaries and venoles. ⁵Without knowing the blood flow, it does not tell us much about oxygen consumption of the tissue (extraction).

The absorption spectrum of a chromophore, a colored molecular constituent is typically shown as a graph of its attenuation as a function of light wavelength. Besides, Hb, HbO₂ and HbCO as shown in FIG. 1, there are many other clinically interesting chromophores found in tissue including methemoglobin, sulfhemoglobin, various cytochromes (distinguished by spectroscopy), bilirubin and glucose.

Note that hemoglobins are strongly absorbing at visible wavelengths—which is why they look colored to our eyes. Water looks transparent (clear) having little absorption in the visible. On the other hand, both water and fat have complex absorption spectra in the infrared.

If tissue were to contain only a mixture of HbO₂ and Hb (in blood)—and no other chromophores—then measurements at only two wavelengths would suffice to determine their relative concentrations. Two wavelengths are commonly used in commercial “pulse oximeters.” If carboxyhemoglobin (HbCO) is present, they will have errors in their readings of oxygen saturation. The latest pulse oximeters correct for carboxyhemoglobin by adding spectral measurements at additional wavelengths.

Wavelength selection in these instruments is by the use of wavelength-selective filters with narrow “pass bands.” These filters are designed to be relatively transparent to a specified wavelength and reject light at all other wavelengths. In order to analyze blood and tissue for an increasing number of constitutes it becomes necessary to add more and more filters with carefully chosen transmission windows. The location of these windows must be such that they maximize sensitivity and discrimination for the desired constituents while minimizing interference from all other constituents.

Therefore, for a two constituent device a minimum of two filters (wavelengths) are needed to generate two equations that can be solved for two unknowns. Three constituents require at least three filters—and so forth. But this only works if these are major constituents and spurious absorption by other chromophores does not materially interfere. The number of filters needed to analyze for many constituents and suppress interference from other chromophores becomes very large.

### OBJECT AND SUMMARY OF THE INVENTION

The present invention solves this wavelength selection issue by using a CCD, a Charge-Coupled capacitor (or other) spectrometer which looks at all wavelengths. This is equivalent to hundreds or thousands of discrete filters. And by using the nose septa as a thin “sample cell” the wavelength range is extended from the near infrared well down into the visible where there is a lot of unique wavelength absorption structure for many chromophores of interest. In all cases the final concentrations of the chromophores will be determined by deconvolving, also known as decomposition, their absorption spectra from the measured total absorption spectra.

Because the body is so transparent, even a very small amount of an absorbing component in the tissue can be measured. How many photons of each wavelength are absorbed by the component depends on its concentration and characteristic absorption spectrum.

For example, if the absorber is the hemoglobin in red cells, the light reduction will depend on the wavelength of the injected photons and the amount of oxygen bound to hemoglobin. The absorption spectrum of hemoglobin is strongly dependent on how much oxygen it is carrying. We are all familiar with the bright red appearance of oxygen-saturated arterial blood compared with the darker and bluer venous blood from which much of the oxygen has been removed. In the infrared, as the photon wavelength gets longer the absorption of oxygen-saturated blood gradually increases while the initially strong absorption of blood with little oxygen falls rapidly. At about 800 nm the curves cross (the “isobestic” point) and at that wavelength the absorption of photons by hemoglobin is uniquely independent of the oxygen carried. By measuring the signal at the isobestic wavelength, we can measure the total concentration of hemoglobin in the region sampled by the photons, and by comparing the absorption at another wavelength we can estimate its average oxygen saturation. This is the principle of operation of the Somanetics INVOS™ Cerebral Oximeter, the first FDA-approved medical device based on this technology (U.S. Pat. No. 4,570,638).

Wherefore it is a primary object of the present invention to probe into the body using only light sources and detectors on the surface of the skin in a simple, non-invasive, cost-effective procedure.

It is also an object of the present invention to overcome the above mentioned shortcomings and drawbacks associated with the prior art.

Another object of the present invention is to provide a relatively simple and effective non-invasive apparatus for determining the concentrations of blood constituents and in particular the concentration of CO in an animal or human subject.

A further object of the present invention is to apply light to a desired tissue sample having a high concentration of blood and determine the amount of the light which is absorbed by the tissue sample.

Yet another object of the present invention is to provide a light source which can impart a plurality of sequentially different light spectra or a “white” light source using a spectrometer for detection. The purpose in either case is to produce a comparison of the spectra with the light passing through the tissue sample.

A still further object of the present invention is to provide a method of determining the CO level in an animal or human subject by applying the light to the nasal septum of the subject.

A still further object of the invention is to provide a method of assembling a catalog of chromophore spectra from the recorded light intensity at each and substantially every wavelength in the spectral range of the spectrometer for each of a plurality of chromophores separately by measuring each wavelength individually at the determined concentration and recorded wavelength and evaluating the spectral shape to determine the spectral signature of a chromophore.

A still further object of the invention is to provide a method to determine the concentration of a plurality of chromophores by measuring the light intensity at each and substantially every wavelength in the spectral range of the spectrometer for each of the plurality of wavelengths and deconvolving the total absorption spectra to determine a relative contribution of each of the plurality of chromophores in the blood of a human or animal subject; and identifying at least one chromophore and its concentration in the blood of the human or animal subject from catalog of chromophore spectra.

The present invention relates to a method for non-invasively determining the relative amounts of a plurality of chromophores in the blood of a human or animal subject, the method comprising the steps of imparting non-continuously a wavelength range of light comprising visible and near-infrared components extending down to at least about 450 nm to a light delivery fiberoptic; passing the wavelength range of light through a selected tissue sample of the nasal septum of the human or animal subject and receiving the spectral absorption portion of the wavelength range of light in a light collecting fiberoptic; providing a spectrometer for receiving the spectral absorption portion of the wavelength range of light from the light collecting fiberoptic; acquiring a first spectrum of the wavelength range of light; acquiring a second spectrum of ambient light at a first predetermined time interval from the acquisition of the first spectrum; acquiring a third reference spectrum of light from the wavelength range of light at a second predetermined time interval from the acquisition of the second spectrum; and acquiring a fourth spectrum of ambient light at a third predetermined time interval from the acquisition of the reference spectrum; subtracting an average of the second and fourth ambient light spectrum from each of the first and third spectrum to determine a total absorption spectra of the selected tissue sample for a plurality of wavelengths selected from the wavelength range of light; and recording light intensity at each and substantially every wavelength in the spectral range of the spectrometer for each of the plurality of wavelengths; performing decomposition of the total absorption spectra by comparing light intensity at a first wavelength to light intensity at adjacent wavelengths to determine the concentration of the plurality of chromophores; assembling a catalog of chromophore spectra from the recorded light intensity at each wavelength in the spectral range of the spectrometer for each of the plurality of chromophores separately by measuring each wavelength individually at the determined concentration and recorded wavelength to evaluate the spectral shape to determine the spectral signature for the chromophore; deconvolving the total absorption spectra to determine a relative contribution of each of the plurality of chromophores in the blood of a human or animal subject; and identifying at least one chromophore and its concentration in the blood of the human or animal subject from the catalog of chromophore spectra.

The method further comprises the steps of impinging the selected tissue surface portion with broadband infrared light; determining a first absorption value of the light in the hemoglobin of the arterial blood flow through the selected tissue sample and comparing to at least a second absorption value determined at a different time from the first absorption value, and evaluating the rate of change between the first absorption value and the second absorption value. The method may further comprise the step of determining the relative percentages of oxyhemoglobin, carboxyhemoglobin and deoxyhemoglobin in the blood in accordance with multiple wavelength absorption values. Performing the deconvolution of the total absorption spectra from the spectrometer in the method uses a technique of developing a matrix-vector notation;
(A+γI)c=b where A is the N×N matrix with elements a_{m,n} given by
a_{m,n}=∫σₘ(λ)σₙ(λ)dλ; I is the N×N identity matrix, c is the N-component vector whose elements are the unknown concentrations and b is the N-component vector with elements bₘ given by bₘ=∫σₘ(λ)F(λ)dλ;
the concentration c which solves (A+γI)c=b is formally given by c=(A+γI)⁻¹b the result of which determines the quantities or concentrations of chromophores in the blood of a human or animal subject.

The present invention is also related to an apparatus for non-invasively determining the relative amounts of a plurality of chromophores in the blood of a human or animal subject comprising a light source imparting a wavelength range including visible and broadband infrared light to a light delivery fiberoptic; a light emission timing disk having an opening positioned between the light source and the light delivery fiber optic; a light collecting fiberoptic for receiving the wavelength range of light having passed through a selected tissue sample of the human or animal subject at a predetermined time interval according to the light emission timing disk; and a spectrometer for measuring the intensity of substantially all wavelengths of light received by the light collecting fiberoptic; and wherein a decomposition of a total absorption spectra of the measured intensity of substantially all wavelengths of light is performed through a comparison of intensity at a first wavelength to an intensity at one or more adjacent wavelengths to determine the concentration of a plurality of chromophore spectra and a catalog of this chromophore spectra is assembled with the determined concentration and wavelength to evaluate the spectral shape to determine the spectral signature for the chromophore; and at least one chromophore and its concentration in the blood of the human or animal subject from the catalog of chromophore spectra is identified. The light source of the apparatus may impart short intense pulses of visible and infrared light to the transmitting fiberoptic. The broadband light imparted by the light source may be in the range of at least about 450 nanometers to 650 nanometers. The light delivery fiberoptic and the light collecting fiberoptic of the apparatus may have free ends maintained in a substantially parallel relationship to define a tissue sample receiving space between the free ends of the fiberoptics. An end surface of the light delivery fiberoptic is formed at about a 45 degree angle relative to the longitudinal axis of the fiberoptic, and an end surface of the light collecting fiberoptic is also formed at about a 45 degree angle, and both end surfaces are provided with a reflective coating thereon so that the light is directed perpendicularly through the tissue sample. The light source of the apparatus may comprise an incandescent lamp which is pulsed by discharging a capacitor through a filament of the lamp, a short-arc xenon lamp and shutter and the light source imparting the visible and infrared light to the light delivery fiberoptic may further comprise a reference fiberoptic connected directly between the light emission timing disk and the spectrometer. The light source of the apparatus imparts a plurality of sequentially different light spectra to the spectrometer within a desired time period. The apparatus may further comprise means for measuring an ambient light spectra sequentially with the light from the light delivery fiberoptic and the light from the reference fiberoptic to the spectrometer. The deconvolution of the total absorption spectra from the spectrometer of the apparatus is performed using a technique of developing a matrix-vector notation;

(A+γI)c=b (17)

where A is the N×N matrix with elements a_{m,n} given by

a_{m,n}=∫σₘ(λ)σₙ(λ)dλ (14);

I is the N×N identity matrix, c is the N-component vector whose elements are the unknown concentrations and b is the N-component vector with elements bₘ given by

bₘ=∫σₘ(λ)F(λ)dλ (15);

the concentration c which solves

(A+γI)c=b (17)

is formally given by

c=(A+γI)⁻¹b (18)

the result of which determines the quantities or concentrations of chromophores in the blood of a human or animal subject.

These and other features, advantages and improvements according to this invention will be better understood by reference to the following detailed description and accompanying drawings.

### BRIEF DESCRIPTION OF THE DRAWINGS

The invention will now be described, by way of example, with reference to the accompanying drawings in which:

FIG. 1 shows the absorption curves for deoxyhemoglobin (Hb), oxyhemoglobin (HbO2), and carboxyhemoglobin (HbCO);

FIG. 2 shows an embodiment of a carboxyhemoglobin detector, according to the present invention;

FIG. 3 shows the ability of a monitor to follow the growth of carboxyhemoglobin in a large (121 lb) pig;

FIG. 4 is a schematic of an embodiment of a more sophisticated spectrometer-based technology system, according to the present invention;

FIGS. 5A-5E are spectral ratios recorded with a spectrometer as in FIG. 4

FIG. 6 is a diagram of the absorption spectra from several subjects (displaced vertically for clarity); and

FIG. 7 is a diagram of absorption spectra of various species of hemoglobin in the wavelength range of 475 to 640 nm.

### DETAILED DESCRIPTION

At any moment, the body's total supply of oxygen is only about 1 gram (just enough to last about four minutes). The length of time that cells can survive following interruption of oxygen depends on the type of tissue. Brain neurons are unrecoverable after only a few minutes. The organs of the body get their supply of oxygen from circulating blood where it is bound to hemoglobin. Hemoglobin is a large protein carried by blood's red cells (erythrocytes) giving them their characteristic color.

Blood consists of 55% plasma (water, dissolved salts and proteins), 45% red cells and a smattering of white cells. There are about 280 million hemoglobin molecules within each of the 10¹³ erythrocytes circulating in the blood. Each molecule of hemoglobin can carry as many as four molecules of oxygen and can transport over 70 times more oxygen than can be simply dissolved in the blood. Virtually all the oxygen carried by the blood is bound to hemoglobin in the red cells.

The lungs supply hemoglobin with oxygen. When hemoglobin is saturated with oxygen, it is called oxyhemoglobin and has the bright red characteristic of arterial (supply veins) blood. The oxygen needed for supplying energy to organs is extracted from the hemoglobin as it passes through their capillary beds. Stripped of oxygen, the hemoglobin becomes darker and takes on the “bluish” color we associate with venous (draining veins) blood. This is called deoxyhemoglobin and is returned to the lungs by the heart and absorbs a fresh supply of oxygen.

Hemoglobin has even more affinity for carbon monoxide (CO) than for oxygen. A heavy smoker will bind enough CO to his hemoglobin to lower the O₂ concentration in the arterial blood by 20%. More fire victims die from a buildup of carbon monoxide in the blood, which displaces oxygen, than all other causes. The greatest threat to life from fires is not the heat or lack of oxygen in the air, but suffocation from the displacement of oxyhemoglobin (HbO₂) with carboxyhemoglobin (HbCO). Carbon monoxide becomes so tightly bound to hemoglobin that the half-life for CO washout after exposure is removed is five hours.

Besides oxyhemoglobin, deoxyhemoglobin and carboxyhemoglobin, there are still other varieties found in special circumstances, in methemoglobin the normal ferrous iron is replaced by ferric iron and occurs in the blood of people poisoned by nitro and amide compounds. Clinically, it is seen in small amounts after the therapeutic use of sulfonamide, nitrates, methylene blue, sulfonal, KCl, etc. Sulfhemoglobin apparently arises from methemoglobin and is more frequently found postmortem. The absorption of light by hemoglobin depends both on the wavelength of the incident light and the kind of hemoglobin. This unique spectral “signature” allows the simultaneous sorting out of different forms of hemoglobin.

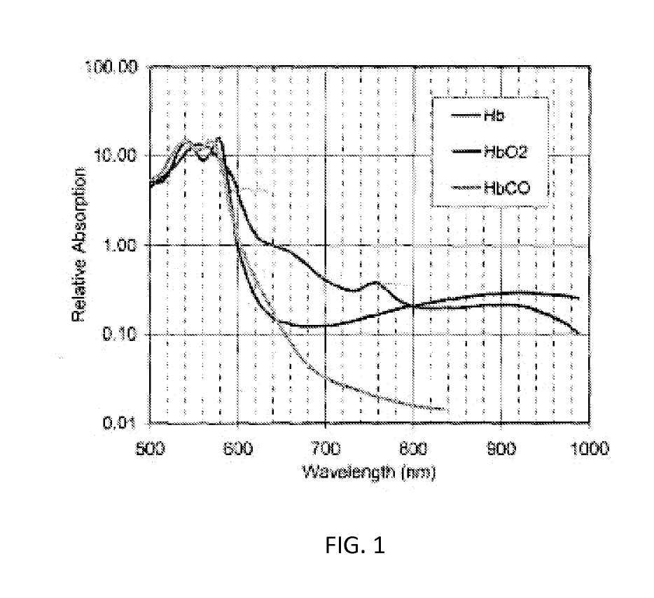

The absorption curves for deoxyhemoglobin (Hb), oxyhemoglobin (HbO₂), and carboxyhemoglobin (HbCO) are shown in FIG. 1. HbCO is far more transparent than Hb or HbO₂ in the near infrared. In principle, measurements of absorption at least three wavelengths, between 500 to 650 nm, will be sufficient to determine relative amounts of each of these three hemoglobin varieties (Other, non-hemoglobin absorption such as water fat and variable tissue scattering make it desirable to use additional wavelengths.)

While spectral measurements can be made in different extremities of a body, an important aspect of the present invention is the choice of the septum of the nose as a point of measurement. There is a high concentration of blood in this area and critically, it is not affected by the shutdown of blood flow to the extremities. An important additional feature is that the septum is thin enough to extend the wavelengths measured below 650 nm where additional spectral signatures are available.

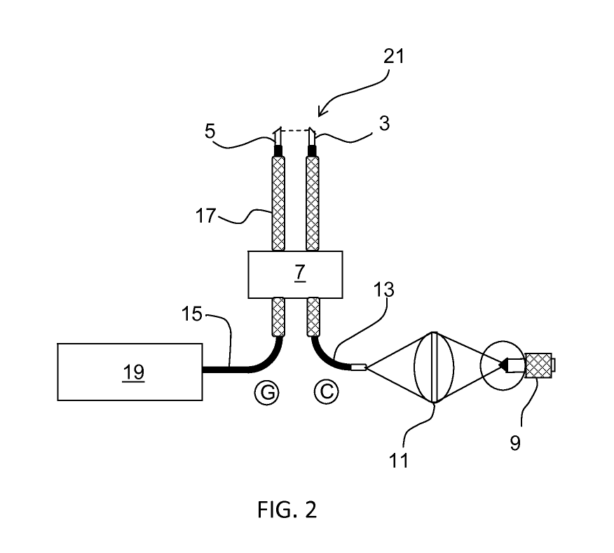

Observing FIG. 2, the transmission spectrum of the septa is measured by inserting two, small, very flexible fiberoptic probes 3, 5, one into each nostril. They are attached to a common base 7 where they are separated by about 8 mm. It is quite easy to insert the fiberoptic probes into the human nose as they are so small as not to affect the airway for normal breathing. The combination of the base 7 with probes is cheap and disposable. They can be incorporated in a mask or presented in other ways. Broadband infrared light is directed into one nostril and the spectral distribution of the light emerging form the other nostril is measured. In the simplest embodiment, broadband near infrared light is produced by the incandescent lamp 9. Light from the filament of the lamp is collected over a large solid angle by lens 11 and focused into the delivery fiberoptic 13. The collecting fiberoptic 15 is held parallel to the delivery fiber by base 7. In addition, both fiberoptics 13, 15 are stiffened by thin aluminum sleeves 17. The collected light is delivered to the CCD (Ocean Optics) spectrometers 19 and 20.

Jacketed 2 mm diameter plastic fibers 13, 15 are used giving an overall diameter of 3 mm. The outside diameter of the aluminum sleeves 17 is 4 mm and the fibers 13, 15 are spaced 12 mm on center giving a separation of 8 mm. By sliding the block 7 up and down, the penetration distance up the nostrils can be set.

An important consideration is the treatment of the (nostril) ends of the delivery and receiving fibers 13, 15 defining the flexible fiberoptic probes 3, 5, so that the light is directed perpendicularly through the septum of the nose. The present method is to cut the ends at 45 degrees to form an angled end surface 21 and cement on a bit of polished aluminum foil or other such reflective surface material onto the end surface 21. it is also possible to provide means for sending some of the light to a silicon photodiode to monitor lamp intensity and to the spectrometer to monitor the lamp spectral distribution.

The best signal-to-noise ratios can be obtained by injecting light in short, intense pulses rather than continuous illumination so uncomfortable heating of the nose with the use of a continuous incandescent lamp is avoided. For example, an incandescent lamp may be pulsed by discharging a capacitor through its filament. Flashing also has the potential for delivering high peak power without burning. The most recent embodiment uses “spectrally tailored” light source with enriched emission in the spectral range of interest achieved with a combination of a short-arc xenon lamp and appropriate colored filters. A spinning disk acts as a shutter and makes it possible to interlace a lamp background spectrum with the transmission spectrum.

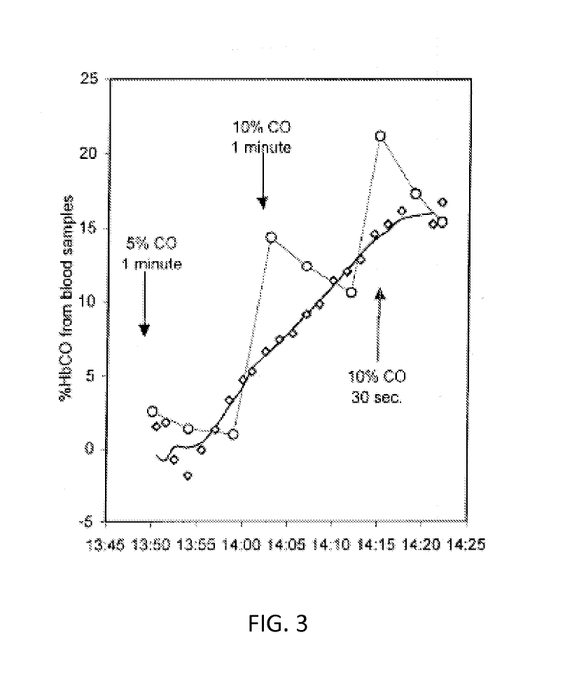

Drs. Stahl and Soller, Department of Surgery at the University of Massachusetts Medical Center tested the Carboxyhemoglobin Detector in an animal model by gradually increasing the CO load of the hemoglobin and comparing the results with Co-oximeter measurements of HbCO from blood samples. FIG. 3 shows the ability of the monitor to follow the growth of carboxyhemoglobin in a large (121 lb) pig. The data, shown as open diamonds, were recorded by the Carboxyhemoglobin Detector at intervals starting at 13:46 and continuing through 14:22. During this time carbon monoxide was momentarily added to the inhaled gas mixture three times. The larger open circles show the HbCO as measured by the University of Massachusetts' co-oximeter using collected samples of arterial blood.

The HbCO concentration in the arterial samples jumps immediately after CO gas is added and then falls as it equilibrates with the total circulating blood volume. Not only does the carboxyhemoglobin detector follow the equilibrium values for the arterial blood samples as measured by the co-oximeter, but also the curve appears to be a bit steeper at about 7 minutes after each inhalation of CO. This curve could be plotted upside down showing the fall in oxyhemoglobin as its oxygen is displaced by carbon monoxide with time.

One particular problem addressed by the present invention is the method of determining the quantities or concentrations of chromophores such as CO in the blood sample. This problem is solved by use of the spectrometer and deconvolution, or decomposition, techniques explained below. Decomposition is best understood as a method of untangling, identifying and determining the concentrations of the various chromophores that are constituents of a blood sample from a composite absorption spectrum obtained by the use of a spectrometer. The absorption spectrum of light which has been passed through the septum of the nasal passages is a specific example. The absorption spectrum recorded by the spectrometer consists of the measured light intensity at each wavelength in the spectral range of the spectrometer. Wavelengths for which the blood sample is absorbing will produce the least light with smallest values in the spectrum while wavelength regions where the blood sample is transparent will produce the largest.

The spectrometer records the composite absorption spectrum of light resulting from the sum of individual absorption spectra of chromophores in a blood sample. What is not immediately known from this composite spectrum is the relative contribution of each of the individual chromophores.

In order to identify the individual chromophores and determine their concentrations we must first know their absorption spectral signatures as a function of concentration. This catalog of chromophore spectra can be assembled by measuring the spectrum of each separately, one-by-one, at a known concentration. In general, the spectral “shape” will provide a unique signature for each chromophore while the actual amount of light absorption will depend on their concentrations.

In the simplest case of two chromophores, e.g. hemoglobin and oxygenated hemoglobin, absorption measurements at only two wavelengths, for which the spectral signatures are different, are required. This leads to two independent equations which can be solved for the concentrations of the two constituents. Pulse oximeters measure oxygen saturation of arterial blood using two optical filters that pass light in two defined wavelength bands. If carboxyhemoglobin is added to the list of unknowns, then three filters are required to provide three equations to solve to obtain the three chromophore concentrations. The problem in these cases is errors caused by ignoring the absorption of other constituents in the substrate—such as fat and water.

The errors caused by the substrate increase as measurement of additional chromophores at increasingly smaller concentrations is attempted. Not only are more optical filters needed for the new constituents, but additional filters are required to deal with the substrate spectral absorption. It becomes increasingly difficult to devise a finite filter set that leads to a sufficiently independent set of equations.

The use of a spectrometer (essentially an array of many adjacent filters) minimizes these problems by measuring the recorded light intensity at each and substantially every wavelength in the spectral range of the spectrometer and producing a large set of equations to solve for a lesser number of chromophores. A matrix can be set up and solved for the constituents with a computer. The only limits to accuracy are system noise in the composite spectrum, missing chromophores in the catalog used for setting up the equations, and lack of independence of the spectral signatures.

More specifically, the constituents are solved by the following method. The intensity of light I(λ) received after passing through a sample with spectral absorption coefficient α(λ) is given by

I(λ)=I₀(λ)e^{-α(λ)l(λ)} (1)

where I₀(λ) is the illumination intensity and l(λ) is the distance photons travel through the sample. In the simplest case, this distance is just the thickness of the sample, but, more generally, it might depend on wavelength if there is sufficient scattering within the sample.

Define sample transmission T(λ) in the usual manner as the ratio

$$T ( λ ) = \frac{I ( λ )}{{I}_{0} ( λ )} = {e}^{- α ( λ ) 𝓁 ( λ )} \qquad ( 2 )$$

this can be expressed in the simpler logarithmic form

−ln T(λ)=α(λ)l(λ) (3)

Total absorption is due to a collection of different absorbing species (chromophores). Assuming we know the absorption cross sections σ₁(λ), σ₂(λ) . . . σ_{N}(λ) of N of these, we can write

$$\begin{matrix}α ( λ ) = {\sum }_{n = 1}^{N} {α}_{n} ( λ ) + {α}_{u} ( λ ) \\ where & ( 4 ) \\ {α}_{n} ( λ ) = {c}_{n} {σ}_{n} ( λ ) & ( 5 )\end{matrix}$$

Any residual absorption not included in the known set is included in the term αᵤ(λ). The coefficients c₁,c₂ . . . c_{N} appearing in (5) are the unknown concentrations of the N chromophores. The values of these are what we are interested in determining and the procedure to do so is described next.

First, substitute (4) and (5) into (3) to obtain

$$- ln T ( λ ) = ( {\sum }_{n = 1}^{N} {c}_{n} {σ}_{n} ( λ ) + {α}_{u} ( λ ) ) 𝓁 ( λ ) \qquad ( 6 )$$

Define F(λ) as

$$F ( λ ) = \frac{- ln T ( λ )}{𝓁 ( λ )} = {\sum }_{n = 1}^{N} {c}_{n} {σ}_{n} ( λ ) + {α}_{u} ( λ ) \qquad ( 7 )$$

Rearrangement of this expression to isolate the unknown absorption coefficient produces

$${α}_{u} ( λ ) = F ( λ ) - {\sum }_{n = 1}^{N} {c}_{n} {σ}_{n} ( λ ) \qquad ( 8 )$$

square both sides and integrate over the measured wavelength domain to obtain

$$\int {α}_{u}^{2} ( λ ) d λ = \int {( F ( λ ) - {\sum }_{n = 1}^{N} {c}_{n} {σ}_{n} ( λ ) )}^{2} d λ \qquad ( 9 )$$

The goal is to find the c₁,c₂ . . . c_{N} which minimize the integrated square residual absorption as defined by the LHS of the above equation. This, of course, is the same as minimizing the RHS. To keep the solution from diverging when σ₁(λ),σ₂(λ) . . . σ_{N}(λ) are weakly independent, we condition the solution by adding a regularizing term that penalizes large coefficients. Hence, we seek to minimize

$$\int {( F ( λ ) - {\sum }_{n = 1}^{N} {c}_{n} {σ}_{n} ( λ ) )}^{2} d λ + γ {\sum }_{n = 1}^{N} {c}_{n}^{2} \qquad ( 10 )$$

with respect to each of the c₁,c₂ . . . c_{N}. The factor γ may be thought of as a Lagrange multiplier that sets the amount of penalty for large coefficients. By definition, at minimum, the derivatives of (10) with respect to each c₁,c₂ . . . c_{N} vanishes, that is

$$\frac{\partial }{\partial {c}_{m}} ( \int {( F ( λ ) - {\sum }_{n = 1}^{N} {c}_{n} {σ}_{n} ( λ ) )}^{2} d λ + γ {\sum }_{n = 1}^{N} {c}_{n}^{2} ) = 0 \qquad ( 11 )$$

for every m. This gives rise to the set of N linear equations

$$\int {σ}_{m} ( λ ) ( F ( λ ) - {\sum }_{n = 1}^{N} {c}_{n} {σ}_{n} ( λ ) ) d λ + γ {c}_{m} = 0 \qquad ( 12 )$$

which, after exchanging the order of integration and summation, become

$${\sum }_{n = 1}^{N} {c}_{n} ( \int {σ}_{m} ( λ ) {σ}_{n} ( λ ) d λ + {γδ}_{m , n} ) = \int {σ}_{m} ( λ ) F ( λ ) d λ \qquad ( 13 )$$

Let

a_{m,n}=∫σₘ(λ)σₙ(λ)dλ (14)

and

bₘ=∫σₘ(λ)F(λ)dλ (15)

Then (13) becomes

$${\sum }_{n = 1}^{N} ( {a}_{m , n} + {γδ}_{m , n} ) {c}_{n} = {b}_{m} \qquad ( 16 )$$

which, in matrix-vector notation, is just

(A+γI)c=b (17)

where A is the N×N matrix with elements a_{m,n} given by (14), I is the N×N identity matrix, c is the N-component vector whose elements are the unknown concentrations and b is the N-component vector with elements bₘ given by (15).

The c which solves (17) is formally given by

c=(A+γI)⁻¹b (18)

If the catalog of chromophores is complete and σ₁(λ),σ₂(λ) . . . σ_{N}(λ) are linearly independent over the domain of measured wavelengths, then the solution to (18) with γ set to zero will be exact and both sides of (9) will vanish. If the catalog is not complete, there will be non-zero residual absorption exhibiting both positive and negative values. Since negative values are not physically possible the accuracy of the solution is improved by increasing γ until αᵤ(λ) is everywhere non-negative. Furthermore, if σ₁(λ),σ₂(λ) . . . σ_{N}(λ) are not completely independent, use of non-zero γ will prevent the solution from diverging when the intensity measurements are noisy.

As the number of absorbers (chromophores) to be monitored increases, more spectral data is needed to sort them out. For developing this application we utilized two Ocean Optics CCD spectrometers, one with a wavelength range of 300 to 1000, and another ranging from 500 to 1200 nm. To deliver as much light as possible, a short (3 mm) arc xenon lamp S was chosen producing the very high brightness needed for getting as much of the light produced into a small fiberoptic cable.

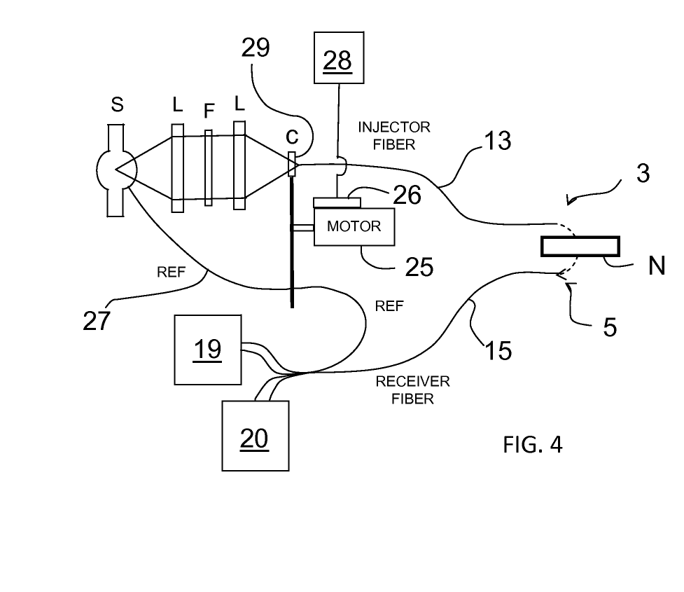

FIG. 4 is a schematic of a more sophisticated system. Light from the short arc xenon flash lamp S is collected and collimated by the f/1 aspheric Fresnel lens L. An identical lens L refocuses the light into the injector fiberoptics. The space provided between the two Fresnel lenses may be used for filters F. After transmission from the delivery fibers 13 to the receiving fibers 15 through the nose septum N, light is conducted to the two CCD spectrometers, the first spectrometer 19 in a range of 300 to 1000, and the second spectrometer 20 in a range from 500 to 1200 nm to record the absorption spectrum of the septum N. A separate reference fiber 27 records the lamp S spectra. The motor 25 drives a “chopper” disk C described below is seen at the center. The light delivery fiber 13 leads to the probe end 5 for being inserted in a subject nasal passage. The reference fiber 27 extends past the disk C and directly to the spectrometers 19 and 20 without passing through the nasal septum N. An electronics board 26 is used to control the motor speed from a computer 28. Operation of the system is described in detail below.

The motor-driven “chopper” disk C, with a single small hole 29, selects the source of the light arriving at the spectrometer 23. The disk C makes one rotation in 200 ms during which (4) four spectra S₁, S₂, S₃, and S₄ are acquired at 50 ms intervals. The first spectrum S₁ is acquired from the tissue when the flash lamp is fired at the time the chopper hole is aligned with the injector fiber. The second spectrum S₂ from ambient light is acquired during the next 50 ms during which time the lamp is off. The third spectrum S₃ is acquired when the flash lamp is fired at the time the chopper hole is aligned with the reference fiber 27. Finally, the fourth spectrum S₄ which is again ambient light is taken and is like the first ambient light spectrum. The cycle is then repeated many times gradually building up these four separately-summed spectra in the computer.

Data analysis begins by subtracting the average ambient light spectra S₂,+S₄/z from S₁, the spectrum containing the tissue reflectance spectrum, and S₃, the spectrum containing the flash lamp spectrum. This leaves:

S₁=S·T·F

S₃=S

Where S is the flash lamp spectrum, T is the tissue transmittance, and F is the filter transmittance. Solving for T:

$$T = \frac{{S}_{1}}{{S}_{3} • F}$$

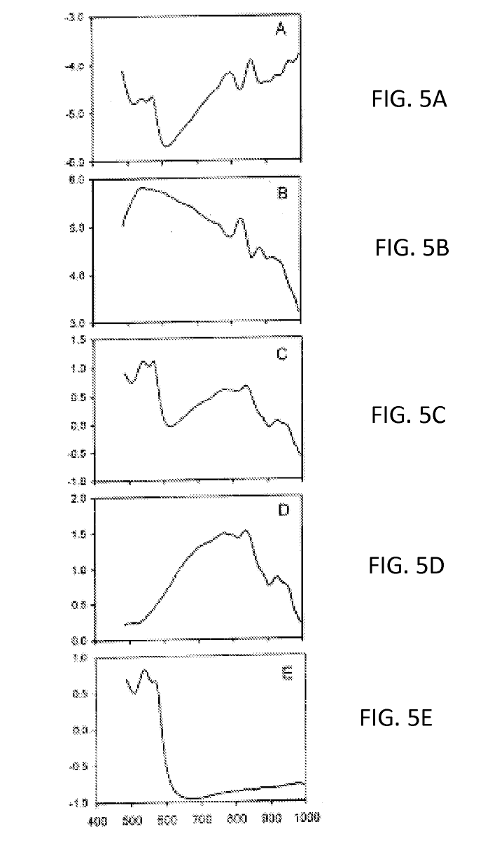

In an experiment, data taken from the nasal septa of a human test subject is shown in FIGS. 5A-5E. Curve A is the reciprocal of S₁. B is S₃ and C is the reciprocal of S₁/S₃. D is the filter absorptance 1/f. E is the resulting absorptance of the nasal septa:

$$A = {\{ \frac{{S}_{1}}{{S}_{3} F} \}}^{- 1}$$

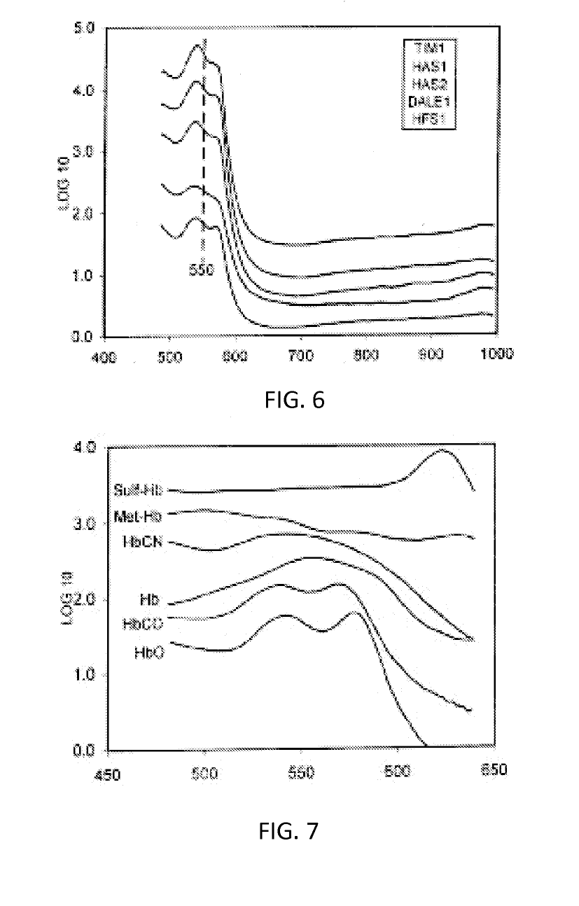

Compare FIG. 5E with the HbO2 curve in FIG. 1. The absorption spectra from five subjects is shown in FIG. 6. The individual spectra have been shifted vertically by 0.4 OD to avoid overlapping.

This technology is an important step in the field of continuous, non-invasive, in vivo monitoring of blood gases. The carboxyhemoglobin detector described above is a subset of this technology. Because of the number of target chromophores, namely, oxy-, deoxy-, carboxy-, cyano-, met- and sulf-hemoglobins, and non-target chromophores, can only be supplied by spectra covering the largest range of wavelengths. The absorption spectra of various species of hemoglobin in the wavelength range of 475 to 640 nm are shown in FIG. 7.

It is especially important to obtain data in the region of 550 nm where hemoglobins have strong absorptance and characteristic spectra. Until now, in vivo NIRS methods have been unable to collect spectral data much below about 650 nm. With the present invention it is possible to extend in vivo spectra down to 450 nm by taking transmission spectra of the thin, blood-rich septum of the nose using high-power, short-arc flash lamps and sophisticated data processing. Also, one or two, 3 mm GB-26 green filters are used to attenuate the large amount of infrared light above 600 nm in order to keep the CCD spectrometer within its dynamic operating range.

Since certain changes may be made in the above described improved method and instrument for measuring carbon monoxide poisoning of humans, without departing from the spirit and scope of the invention herein involved, it is intended that all of the subject matter of the above description or shown in the accompanying drawings shall be interpreted merely as examples illustrating the inventive concept herein and shall not be construed as limiting the invention.

Wherefore, I/We claim:

1. A method for non-invasively determining the relative amounts of a plurality of chromophores in the blood of a human or animal subject, the method comprising the steps of:
imparting non-continuously a wavelength range of light comprising visible and near-infrared components extending down to at least about 450 nm to a light delivery fiberoptic;
passing the wavelength range of light through a selected tissue sample of the nasal septum of the human or animal subject and receiving a spectral absorption portion of the wavelength range of light in a light collecting fiberoptic;
providing a spectrometer for receiving the spectral absorption portion of the wavelength range of light from the light collecting fiberoptic;
acquiring a first spectrum of the spectral absorption portion of the wavelength range of light;
acquiring a second spectrum of ambient light at a first predetermined time interval from the acquisition of the first spectrum;
acquiring a third reference spectrum of light from the wavelength range of light at a second predetermined time interval from the acquisition of the second spectrum; and
acquiring a fourth spectrum of ambient light at a third predetermined time interval from the acquisition of the reference spectrum;
subtracting an average of the second and fourth ambient light spectrum from each of the first and third spectrum to determine a total absorption spectra of the selected tissue sample for a plurality of wavelengths selected from the wavelength range of light; and
recording light intensity at each and substantially every wavelength in the spectral range of the spectrometer for each of the plurality of wavelengths;
performing decomposition of the total absorption spectra by comparing light intensity at a first wavelength to light intensity at adjacent wavelengths to determine the concentration of the plurality of chromophores;
assembling a catalog of chromophore spectra from the recorded light intensity at each wavelength in the spectral range of the spectrometer for each of the plurality of chromophores separately by measuring each wavelength individually at the determined concentration and recorded wavelength to evaluate the spectral shape to determine the spectral signature for the chromophore;
deconvolving the total absorption spectra to determine a relative contribution of each of the plurality of chromophores in the blood of a human or animal subject; and
identifying at least one chromophore and its concentration in the blood of the human or animal subject from the catalog of chromophore spectra.

2. The method for non-invasively determining the relative amount of at least one chromophore in the blood of a human or animal subject as set forth in claim 1 further comprising the step of impinging the selected tissue surface portion with broadband infrared light.

3. The method for non-invasively determining the relative amount of at least one chromophore in the blood of a human or animal subject as set forth in claim 1 further comprising the step of determining a first absorption value of the light in the hemoglobin of the arterial blood flow through the selected tissue sample and comparing to at least a second absorption value determined at a different time from the first absorption value, and evaluating the rate of change between the first absorption value and the second absorption value.

4. The method for non-invasively determining the relative amount of at least one chromophore in the blood of a human or animal subject as set forth in claim 1 further comprising the step of determining the relative percentages of oxyhemoglobin, carboxyhemoglobin and deoxyhemoglobin in the blood in accordance with multiple wavelength absorption values.

5. The method for non-invasively determining the relative amounts of at least one chromophore in the blood of a human or animal subject as set forth in claim 1 further comprising the steps of performing the deconvolution of the total absorption spectra from the spectrometer using a technique of developing a matrix-vector notation;
(A+γI)c=b (17)
where A is the N×N matrix with elements a_{m,n} given by
a_{m,n}=∫σₘ(λ)σₙ(λ)dλ (14);
I is the N×N identity matrix, c is the N-component vector whose elements are the unknown concentrations and b is the N-component vector with elements bₘ given by
bₘ=∫σₘ(λ)F(λ)dλ (15);
the concentration c which solves
(A+γI)c=b (17)
is formally given by
c=(A+γI)⁻¹b (18)
the result of which determines the quantities or concentrations of chromophores in the blood of a human or animal subject.

6. An apparatus for non-invasively determining the relative amounts of a plurality of chromophores in the blood of a human or animal subject comprising:
a light source imparting a wavelength range including visible and broadband infrared light to a light delivery fiberoptic;
a light emission timing disk having an opening positioned between the light source and the light delivery fiber optic;
a light collecting fiberoptic for receiving the wavelength range of light having passed through a selected tissue sample of the human or animal subject at a predetermined time interval according to the light emission timing disk; and
a spectrometer for measuring the intensity of substantially all wavelengths of light received by the light collecting fiberoptic; and
wherein a decomposition of a total absorption spectra of the measured intensity of substantially all wavelengths of light is performed through a comparison of intensity at a first wavelength to an intensity at one or more adjacent wavelengths to determine the concentration of a plurality of chromophore spectra and a catalog of this chromophore spectra is assembled with the determined concentration and wavelength to evaluate the spectral shape to determine the spectral signature for the chromophore; and
at least one chromophore and its concentration in the blood of the human or animal subject from the catalog of chromophore spectra is identified.

7. The apparatus for non-invasively determining the relative amounts of a plurality of chromophores in the blood of a human or animal subject as set forth in claim 6 wherein the light source imparts short intense pulses of visible and infrared light to the transmitting fiberoptic.

8. The apparatus for non-invasively determining the relative amounts of a plurality of chromophores in the blood of a human or animal subject as set forth in claim 6 wherein the broadband light imparted by the light source is in the range of at least about 450 nanometers to 650 nanometers.

9. The apparatus for non-invasively determining the relative amounts of a plurality of chromophores in the blood of a human or animal subject as set forth in claim 6 wherein the light delivery fiberoptic and the light collecting fiberoptic have free ends maintained in a substantially parallel relationship to define a tissue sample receiving space between the free ends of the fiberoptics.

10. The apparatus for non-invasively determining the relative amounts of a plurality of chromophores in the blood of a human or animal subject as set forth in claim 9 wherein an end surface of the light delivery fiberoptic is formed at about a 45 degree angle relative to the longitudinal axis of the fiberoptic, and an end surface of the light collecting fiberoptic is also formed at about a 45 degree angle, and both end surfaces are provided with a reflective coating thereon so that the light is directed perpendicularly through the tissue sample.

11. The apparatus for non-invasively determining the relative amounts of a plurality of chromophores in the blood of a human or animal subject as set forth in claim 6 wherein the light source comprises an incandescent lamp which is pulsed by discharging a capacitor through a filament of the lamp.

12. The apparatus for non-invasively determining the relative amounts of a plurality of chromophores in the blood of a human or animal subject as set forth in claim 6 wherein the light source comprises a short-arc xenon lamp and shutter.

13. The apparatus for non-invasively determining the relative amounts of a plurality of chromophores in the blood of a human or animal subject as set forth in claim 6 wherein the light source imparting the visible and infrared light to the light delivery fiberoptic further comprises a reference fiberoptic connected directly between the light emission timing disk and the spectrometer.

14. The apparatus for non-invasively determining the relative amounts of a plurality of chromophores in the blood of a human or animal subject as set forth in claim 13 wherein the light source imparts a plurality of sequentially different light spectra to the spectrometer within a desired time period.

15. The apparatus for non-invasively determining the relative amounts of a plurality of chromophores in the blood of a human or animal subject as set forth in claim 13 further comprising means for measuring an ambient light spectra sequentially with the light from the light delivery fiberoptic and the light from the reference fiberoptic to the spectrometer.

16. The apparatus for non-invasively determining the relative amounts of a plurality of chromophores in the blood of a human or animal subject as set forth in claim 6 wherein deconvolution of the total absorption spectra from the spectrometer is performed using a technique of developing a matrix-vector notation;
(A+γI)c=b (17)
where A is the N×N matrix with elements a_{m,n} given by
a_{m,n}=∫σₘ(λ)σₙ(λ)dλ (14);
I is the N×N identity matrix, c is the N-component vector whose elements are the unknown concentrations and b is the N-component vector with elements bₘ given by
bₘ=∫σₘ(λ)F(λ)dλ (15);
the concentration c which solves
(A+γI)c=b (17)
is formally given by
c=(A+γI)⁻¹b (18)
the result of which determines the quantities or concentrations of chromophores in the blood of a human or animal subject.
